(19) United States
(12) Patent Application Publication
(10) Pub. No.: US 2020/0317636 A1
(43) Pub. Date: Oct. 8, 2020

(54) DEUTERATED AZOLE COMPOUNDS AND PREPARATION METHOD THEREFOR AND USES THEREOF

(71) Applicants: Tingjunhong NI, Shanghai (CN); Guangqing XU, Shanghai (CN); Yuntao ZHU, Qidong, Nantong (CN); Shuang LI, Shanghai (CN); Jing ZHAO, Lu'an (CN)

(72) Inventors: Tingjunhong NI, Shanghai (CN); Guangqing XU, Shanghai (CN); Yuntao ZHU, Qidong, Nantong (CN); Shuang LI, Shanghai (CN); Jing ZHAO, Lu'an (CN)

(21) Appl. No.: 16/756,083

(22) PCT Filed: Oct. 10, 2018
(86) PCT No.: PCT/CN2018/109738

§ 371 Date: Apr. 14, 2020

(30) Foreign Application Priority Data
Oct. 14, 2017 (CN) 201710955700.X

(51) Int. Cl. C07D 401/06; C07F 9/6524

(52) U.S. Cl. CPC C07D 401/06; C07F 9/6524

(57) ABSTRACT

Disclosed are a deuterated azole compound of formula (I) and a preparation method and an application of a pharmaceutically acceptable salt and prodrug of the compound. The deuterated azole compound disclosed by the invention has good antifungal activity and metabolic stability, and can be used for preparing antifungal drugs.

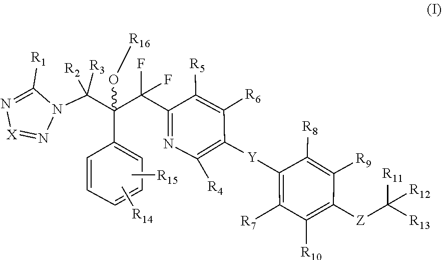

### CROSS-REFERENCE TO RELATED APPLICATION

This application claims priority from Chinese Patent Application No. 201710955700.X, filed on Oct. 14, 2017, in State Intellectual Property Office of P.R.China, the contents of which are hereby incorporated by reference in their entirety for all purposes.

### BACKGROUND OF THE INVENTION

### 1. Technical Field

The invention relates to the synthesis of antifungal drugs, and in particular to a deuterated azole compound, a method for preparing prodrugs and a pharmaceutically acceptable salt of the compound, and an application of the compound in treating or preventing fungal infections.

### 2. Description of Related Art

Deep fungal infections have now become the leading cause of death from major diseases such as AIDS and tumors. However, antifungal drugs currently used in clinical practice have large side effects and a narrow antibacterial spectrum and are prone to drug resistance. Effective antifungal drugs, especially drugs for deep fungal infections, are scarce and can hardly satisfy treatment requirements. Antifungal drugs VT-1161, VT-1129, and VT-1598 developed by VIAMET Corporation in the United States are in the preclinical stage nowadays and have the following structures:

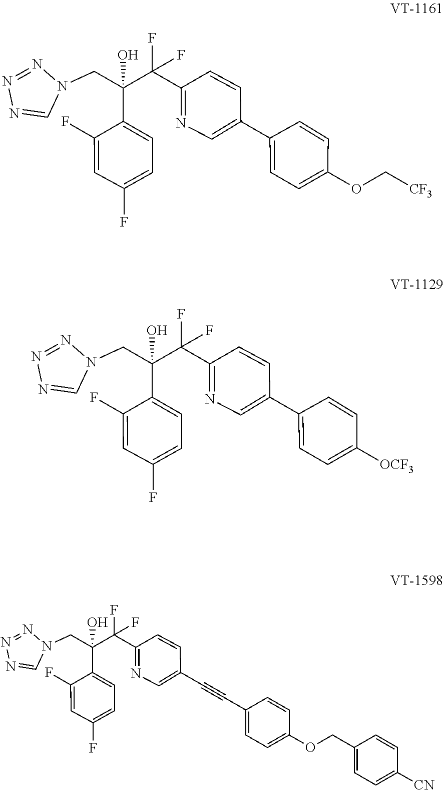 [SMILES: O[C@@](Cn1cnnn1)(c1ccc(F)cc1F)C(F)(F)c1ccc(-c2ccc(OCC(F)(F)F)cc2)cn1 ; O[C@@](Cn1cnnn1)(c1ccc(F)cc1F)C(F)(F)c1ccc(-c2ccc(OC(F)(F)F)cc2)cn1 ; N#Cc1ccc(COc2ccc(C#Cc3ccc(C(F)(F)[C@](O)(Cn4cnnn4)c4ccc(F)cc4F)nc3)cc2)cc1]

These compounds typically act on the target CYP51 of fungal cells and have the advantages of broader antibacterial spectrum, low toxicity, high safety and good selectivity against conventional triazole antifungal drugs. However, VT-1161, VT-1129 and VT-1598 still need to be improved in the aspect of pharmacodynamic and pharmacokinetic properties.

### SUMMARY OF THE INVENTION

In view of the shortcomings in pharmacodynamics and pharmacokinetics in the prior art, an objective of the invention is to provide a deuterated azole compound or a pharmaceutically acceptable salt thereof.

A second objective of the invention is to provide a pharmaceutical composition comprising the deuterated azole compound or the pharmaceutically acceptable salt thereof according to the first aspect.

A third objective of the invention is to provide a preparation method of the deuterated azole compound or the pharmaceutically acceptable salt thereof.

A fourth objective of the invention is to provide an application of the pharmaceutical composition according to the second aspect to the preparation of drugs for treating fungal infections.

To achieve the above objectives, the invention adopts the following technical solutions:

A deuterated azole compound or a pharmaceutically acceptable salt thereof is represented by formula (I):

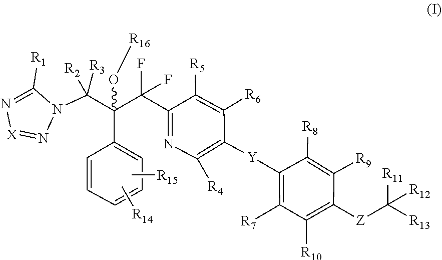

wherein:

R1-R12 are hydrogen atoms or deuterium atoms;

R13 is selected from C1-C6 alkyl, halogen-substituted alkyl, phenyl, pyridyl, C1-6 alkyl-substituted phenyl or pyridyl, halogen-substituted C1-6 alkyl-substituted phenyl or pyridyl, halogen-substituted phenyl or pyridyl, nitro-substituted phenyl or pyridyl, cyano-substituted phenyl or pyridyl, and trifluoromethyl-substituted phenyl or pyridyl, respectively;

R14 and R15 are each selected from a deuterium atom, a hydrogen atom, or a halogen;

R16 is a hydrogen atom or a phosphate group;

X represents N or CH;

Y represents

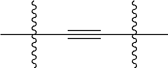 [SMILES: *C#C*]

or non-substituted;

Z represents O or S.

Preferably, the deuterium isotope content of deuterium at the deuterium substitution position is greater than the natural deuterium isotope content (0.015%), preferably by 30%, more preferably by 50%, more preferably by 75%, more preferably by 95%, and more preferably by 99%.

More preferably, the deuterated azole compound is a compound represented by formula (II) or a compound represented by formula (III):

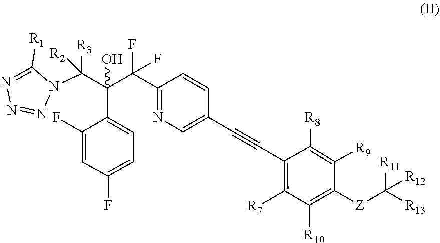

wherein R1-R12 are each selected from a hydrogen atom or a deuterium atom;

R13 is selected from toluene, cyano-substituted phenyl, trifluoromethyl-substituted phenyl, difluoromethyl-substituted phenyl, trifluoromethoxy-substituted phenyl, halogen-substituted phenyl, and cyano-substituted pyridine, and Z is an oxygen atom or a sulfur atom;

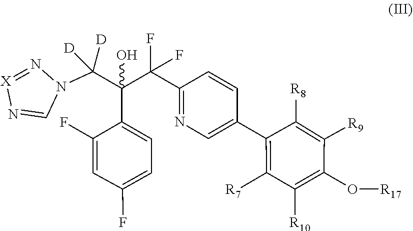

wherein R7-R10 are each selected from a hydrogen atom or a deuterium atom, and R17 is a trifluoromethyl group or a trifluoromethoxy group.

The phenyl or pyridyl in the R13 group described in the aforementioned compound is a deuterated phenyl or a deuterated pyridyl.

The compound includes R-isomers or S-isomers, the R-isomers are represented by:

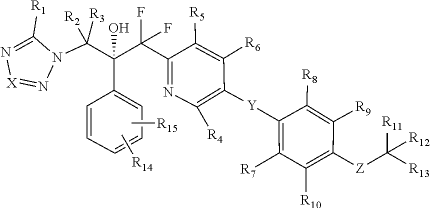

the S-isomers are represented by:

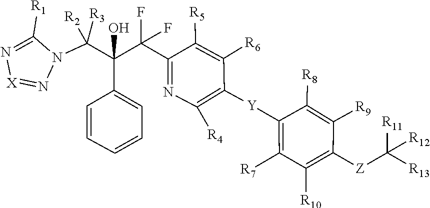

The pharmaceutically acceptable salt is a salt of inorganic acid or of organic acid;

Preferably, the inorganic acid is hydrochloric acid or phosphoric acid; the organic acid is p-toluenesulfonic acid, acetic acid, maleic acid, fumaric acid, tartaric acid, and succinic acid.

The invention further provides a crystalline compound, and the crystalline compound is the deuterated azole compound or the pharmaceutically acceptable salt thereof.

The invention further provides a pharmaceutical composition comprising the deuterated azole compound or the pharmaceutically acceptable salt thereof.

Preferably, the pharmaceutical composition further comprises: at least one pharmaceutically acceptable carrier, or/and at least one additional antifungal compound.

More preferably, the additional antifungal compound includes, but is not limited to, any one or two of clotrimazole, fluconazole, voriconazole, posaconazole, ketoconazole and itraconazole, and a pharmaceutically acceptable salt or ester of the above compound.

To achieve the third objective stated above, the invention adopts the following technical solution:

A preparation method of the deuterated azole compound or the pharmaceutically acceptable salt thereof comprises the following steps:

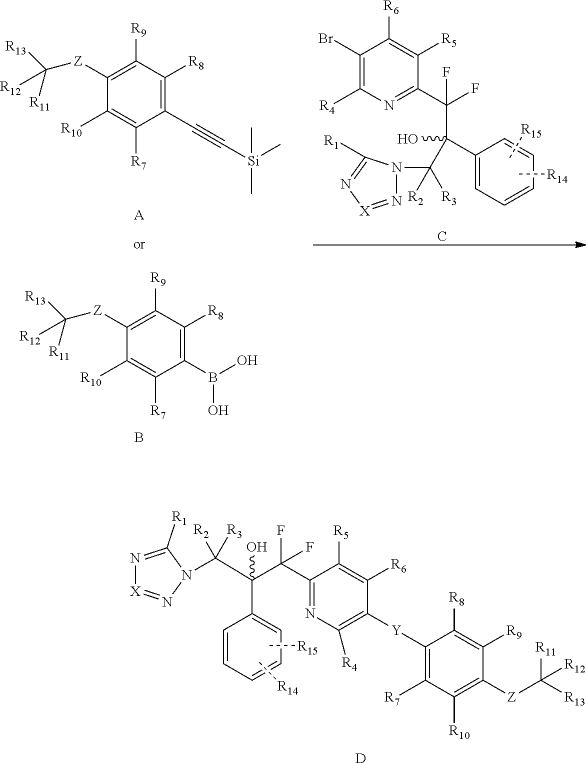

dissolving compound C in an organic solvent, adding a palladium catalyst, an alkali reagent, and compound A or compound B, and heating in the presence of nitrogen to cause a reaction to obtain a target product, i.e., compound D.

Preferably, the organic solvent is N, N-dimethylformamide, tetrahydrofuran, dimethyl sulfoxide, toluene, N-methylpyrrolidone, and dioxane.

Preferably, the palladium catalyst is Pd(PPh₃)₂Cl₂, Pd(PPh₃)₄, Pd(CH₃CN)₂Cl₂, Pd(dppf)Cl₂, or PdCl₂.

Preferably, the alkaline reagent is K₂CO₃, Na₂CO₃, Cs₂CO₃, K₃PO₄, triethylamine, or N, N-diisopropylethylamine.

The invention further provides an application of the pharmaceutical composition in preparing drugs for treating fungal infections.

The invention has the advantages that deuterated azole compound is synthesized; the compound has a good inhibitory activity on the human pathogenic fungus, Candida albicans, and the compound of the invention has significantly better stability to human liver microsomal enzymes than the control compound VT-1598. Moreover, after deuteration, drug metabolism becomes difficult, which leads to a reduction in first-pass effects and a significant increase in drug stability. In this case, the dosage can be changed and a long-acting preparation can be formed; and the applicability may also be improved in the form of a long-acting preparation.

Obviously, based on the above description of the invention, other various modifications, substitutions, or alterations can be made without departing from the basic technical idea of the invention, with reference to common technical knowledge and conventional means in the art.

### DESCRIPTION OF THE PREFERRED EMBODIMENTS

The above content of the invention will be further described in detail below through specific implementations by way of example. However, it should not be understood that the scope of the above subject matters of the invention is limited to the following examples. Any technology implemented based on the above contents of the invention shall fall within the scope of the invention.

The reagents and raw materials used in the invention are all commercially available or can be prepared according to methods from literatures. The experimental methods without specific conditions in the following examples are generally conducted under conventional conditions or conditions recommended by the manufacturer. The reagents used in the examples are all commercially available analytical grade.

### Example 1: Synthesis of Compound D1

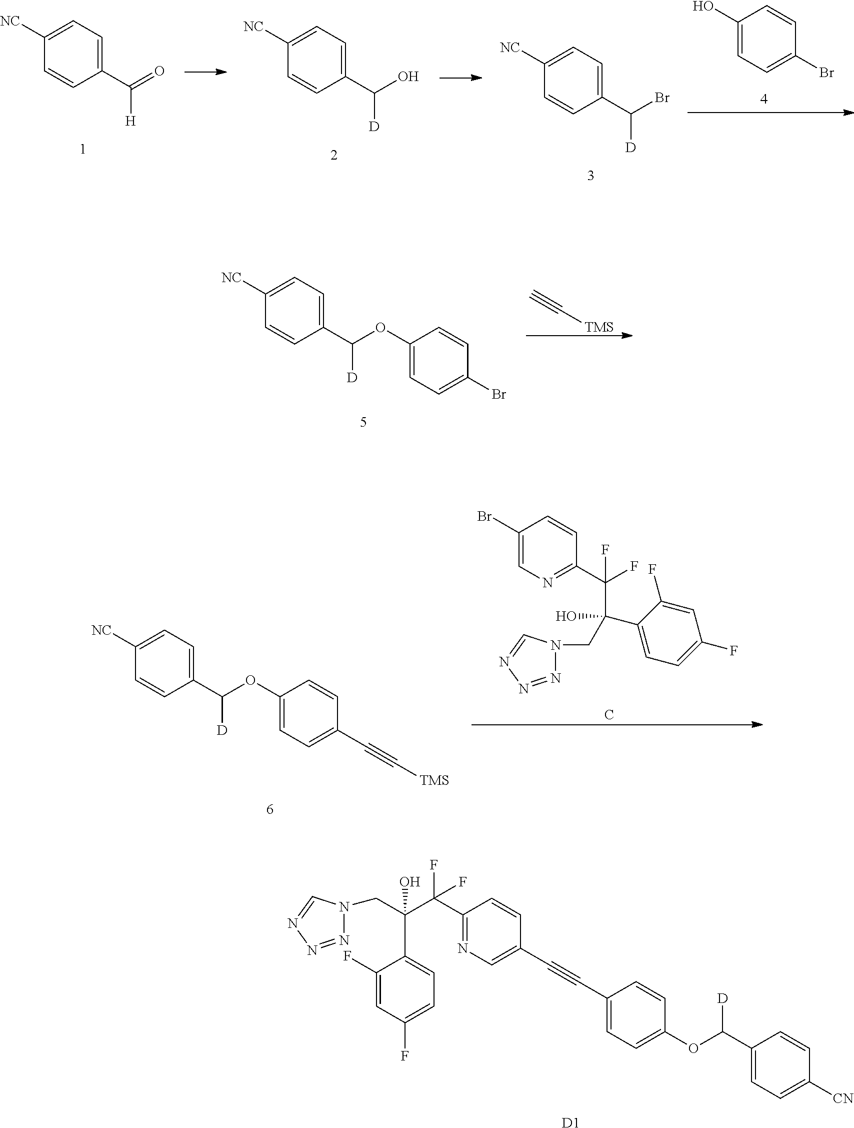

Step 1: compound 1 (5 mmol) was dissolved in tetrahydrofuran, and sodium boron deuteride (3 mmol) was then added under an ice-bath condition. After the raw materials were completely reacted, the reaction solution was poured in ice water and extracted 3 times with ethyl acetate; after being dried, the extract was spin-dried to obtain compound 2, and compound 2 was used directly in the next step.

Step 2: compound 2 (3 mmol) was dissolved in dichloromethane, and phosphorus tribromide (3 mmol) was then added under an ice-bath condition. After the reaction was complete, the reaction solution was poured in ice water and extracted 3 times with dichloromethane; the extract was washed once with 1M diluted hydrochloric acid, once with saturated sodium bicarbonate, once with saturated sodium chloride; after being dried, the extract was spin-dried to compound 3, and compound 3 was used directly in the next step.

Step 3: compounds 3 (2 mmol) and 4 (2 mmol) were dissolved in N, N-dimethylformamide, and potassium carbonate (4 mmol) was then added to react at 50° C. After the reaction was complete, the reaction solution was poured in ice water and extracted 3 times with ethyl acetate; after being dried, the extract was spin-dried and subjected to column chromatography to obtain compound 5.

Step 4: compound 5 (2 mmol), ethynyltrimethylsilane (4 mmol), CuI (5% mol), Pd(PPh₃)₂Cl₂ (10% mol), N, N-diisopropylethylamine (10 mmol) were dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain compound 6.

Step 5: compound C (1 mol), as well as compound 6 (1 mol), CuI (5% mol), Pd(PPh₃)₂Cl₂ (10% mol), N, N-diisopropylethylamine (5 mol), and potassium fluoride (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D1.

¹H NMR (300 MHz, DMSO) δ 9.14 (s, 1H), 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.65-5.60 (d, J=15.0 Hz, 1H), 5.28 (s, 1H), 5.12-5.07 (d, J=15.0 Hz, 1H).

### Example 2: Synthesis of Compound D2

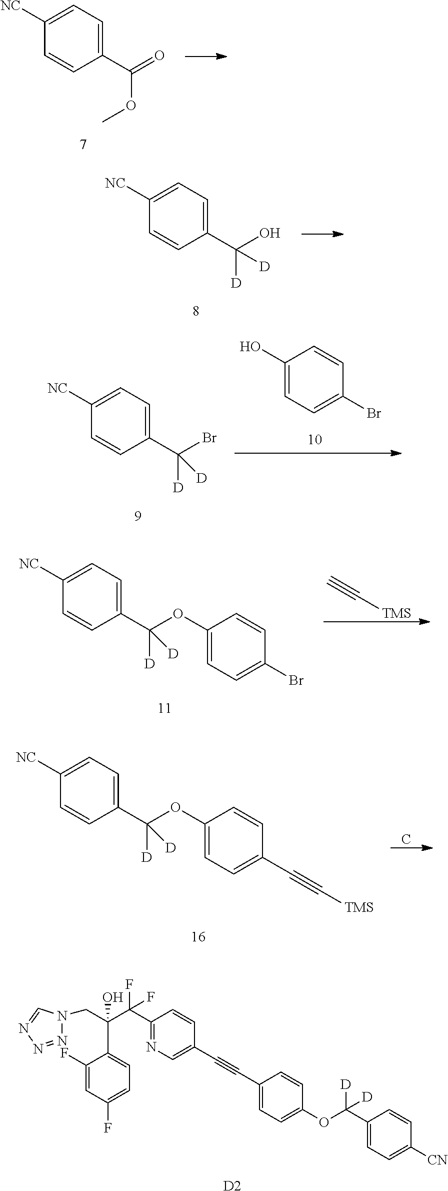

Step 1: compound 7 (5 mmol) was dissolved in tetrahydrofuran, and lithium aluminum deuteride (6 mmol) was then added to react at −78° C. After the raw materials were reacted completely, lithium aluminum deuteride was added in portions at a mass ratio of 1 (H₂O):1 (15% NaOH):3 (H₂O) and stirred for 10 minutes, the solid was filtered, and the filtrate was spin-dried to obtain compound 8. The crude product was used directly in the next step.

Step 2: compound 8 (3 mmol) was dissolved in dichloromethane, and phosphorus tribromide (3 mmol) was then added under an ice-bath condition. After the reaction was complete, the reaction solution was poured in ice water and extracted 3 times with dichloromethane; the extract was washed once with 1M diluted hydrochloric acid, once with saturated sodium bicarbonate, once with saturated sodium chloride; after being dried, the extract was spin-dried to compound 9, and compound 9 was used directly in the next step.

Step 3: compounds 9 (2 mmol) and 10 (2 mmol) were dissolved in N, N-dimethylformamide, and potassium carbonate (4 mmol) and KI (2 mmol) were then added to react at 50° C. After the reaction was complete, the reaction solution was poured in ice water and extracted 3 times with ethyl acetate; after being dried, the extract was spin-dried and subjected to column chromatography to obtain compound 11.

Step 4: compound 11 (2 mmol), ethynyltrimethylsilane (4 mmol), CuI (5% mol), Pd(PPh₃)₂Cl₂ (10% mol), N, N-diisopropylethylamine (10 mmol) were dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain compound 12.

Step 5: compound 12 (1 mol), as well as compound C (1 mol), CuI (5% mol), Pd (PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D2.

¹H NMR (300 MHz, DMSO) δ 9.14 (s, 1H), 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.65-5.60 (d, J=15.0 Hz, 1H), 5.12-5.07 (d, J=15.0 Hz, 1H).

### Example 3: Synthesis of Compound D3

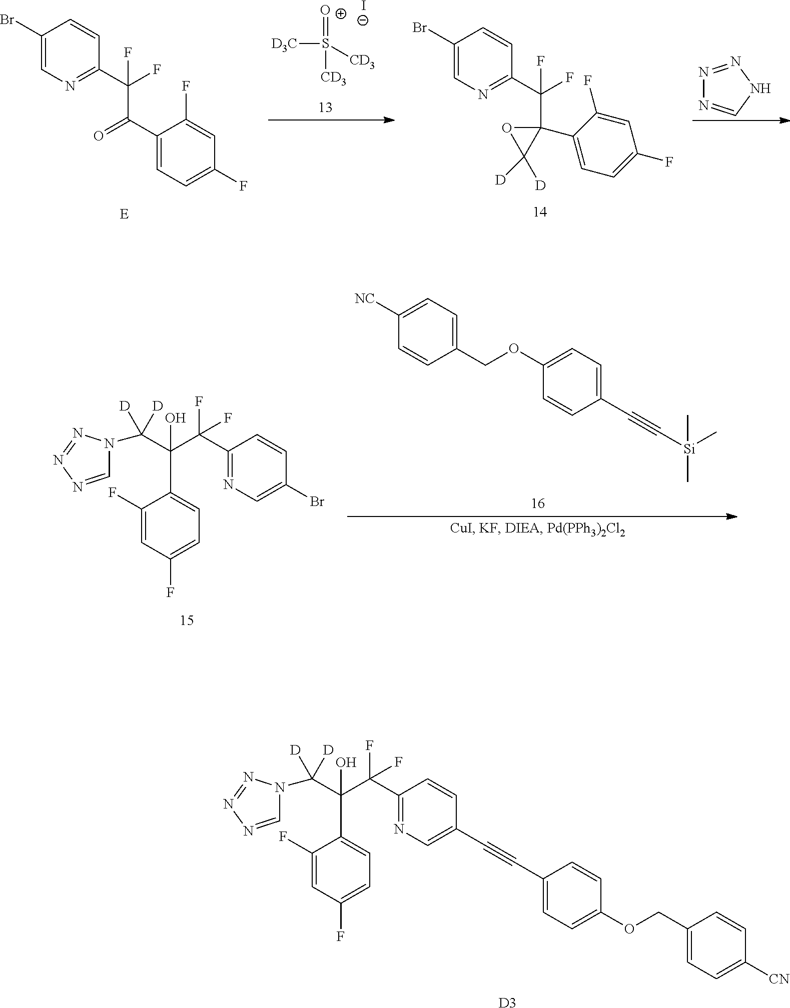

Step 1: 25 g of deuterated methyl iodide and deuterated dimethyl sulfoxide (25 mL) were heated under reflux for 3 days, and a solid was precipitated and filtered to obtain compound 13, and compound 13 was used directly in the next step.

Step 2: compound 13 (2 mmol) was dissolved in anhydrous N, N-dimethylformamide, and sodium hydride (2 mmol) was then added at −5° C.; after stirring for 10 minutes, compound E (2 mmol) was added. After the reaction was complete, the reaction solution was poured into ice water, and extracted 3 times with ethyl acetate; the extract was washed once with saturated sodium chloride; after being dried, the extract was spin-dried and subjected to column chromatography to compound 14, and compound 14 was used directly in the next step.

Step 3: compound 14 (2 mmol) and 1-H-tetrazole (6 mmol) were dissolved in N, N-dimethylformamide, and potassium carbonate (6 mmol) was then added to react at 90° C. After the reaction was complete, the reaction solution was poured in ice water and extracted 3 times with ethyl acetate; after being dried, the extract was spin-dried and subjected to column chromatography to obtain compound 15.

Step 4: compound 15 (1 mmol), as well as compound 16 (1 mmol), CuI (5% mol), KF (1 mmol), Pd(PPh₃)₂Cl₂ (10% mol), and N, N-diisopropylethylamine (5 mol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D3.

¹H NMR (300 MHz, DMSO) δ 9.14 (s, 1H), 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.28 (s, 2H).

### Example 4: Synthesis of Compound D4

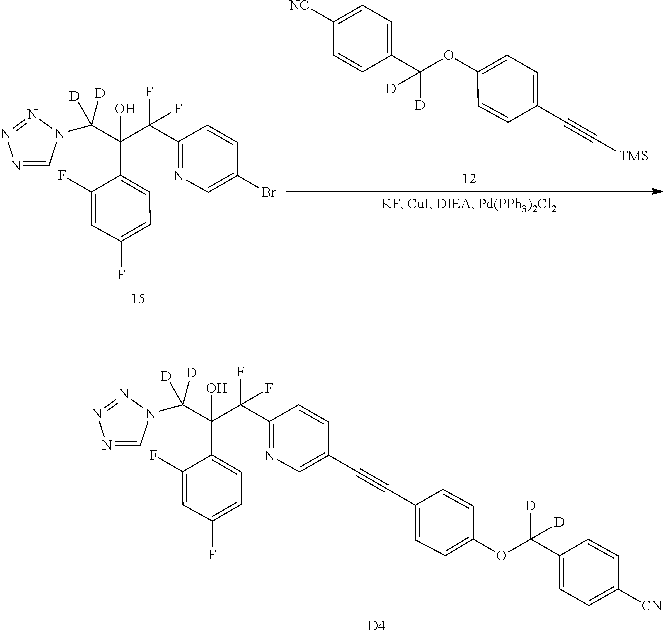 [SMILES: [2H]C([2H])(n1cnnn1)C(O)(c1ccc(F)cc1F)C(F)(F)c1ccc(Br)cn1 ; [2H]C([2H])(Oc1ccc(C#C[Si](C)(C)C)cc1)c1ccc(C#N)cc1 ; [2H]C([2H])(Oc1ccc(C#Cc2ccc(C(F)(F)C(O)(c3ccc(F)cc3F)C([2H])([2H])n3cnnn3)nc2)cc1)c1ccc(C#N)cc1 ; "D4"]

Compound 15 (1 mol), as well as compound 12 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D4.

¹H NMR (300 MHz, DMSO) δ 9.14 (s, 1H), 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H).

### Example 5: Synthesis of Compound D5

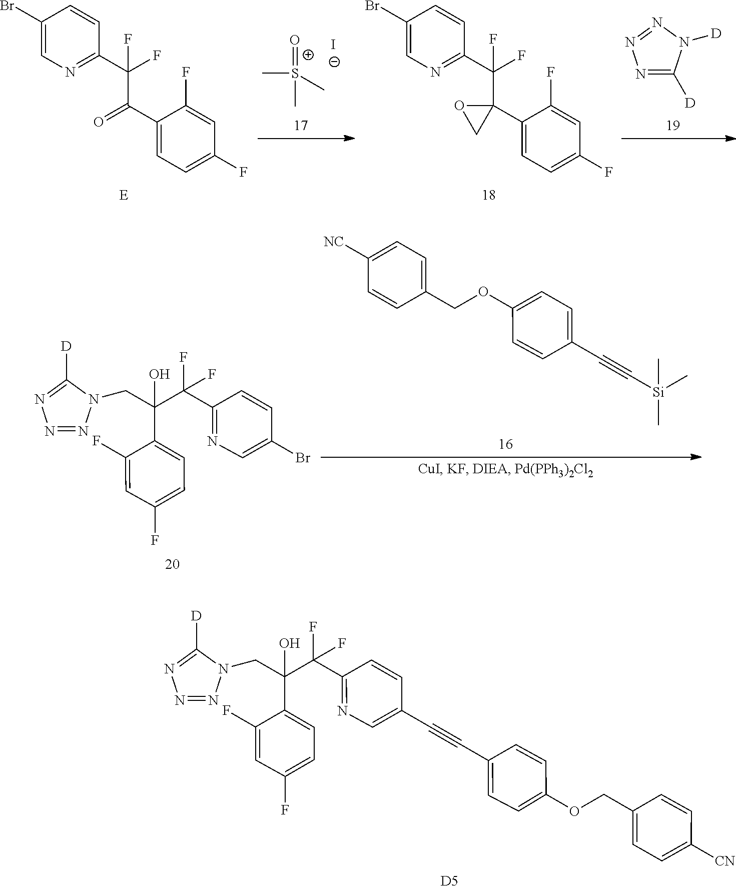

Step 1: compound 17 (2 mmol) was dissolved in anhydrous N, N-dimethylformamide, and sodium tert-butoxide (2 mmol) was then added at −5° C.; after stirring for 10 minutes, compound E (2 mmol) was added. After the reaction was complete, the reaction solution was poured into ice water, and extracted 3 times with ethyl acetate; the extract was washed once with saturated sodium chloride; after being dried, the extract was spin-dried and subjected to column chromatography to compound 18, and compound 18 was used directly in the next step.

Step 2: palladium on carbon (2 g) was stirred in hydrogen for 1 hour, then added to deuteroxide, and then 1-H-tetrazolium was added. After removing the hydrogen, the reaction system was stirred at 100° C. for 1 hour; the palladium on carbon was filtered out and the reaction solution was spin-dried to obtain compound 19, and the crude product was used directly in the next step.

Step 3: compounds 18 (2 mmol) and 19 (6 mmol) were dissolved in N, N-dimethylformamide, and potassium carbonate (6 mmol) was then added to react at 90° C. After the reaction is complete, the reaction solution was poured in ice water and extracted 3 times with ethyl acetate; after being dried, the extract was spin-dried and subjected to column chromatography to obtain compound 20.

Step 4: compound 20 (1 mmol), as well as compound 16 (1 mmol), CuI (5% mol), KF (1 mmol), Pd(PPh₃)₂Cl₂ (10% mol), and N, N-diisopropylethylamine (5 mol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D5.

¹H NMR (300 MHz, DMSO) δ 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.65-5.60 (d, J=15.0 Hz, 1H), 5.28 (s, 2H), 5.12-5.07 (d, J=15.0 Hz, 1H).

### Example 6: Synthesis of Compound D6

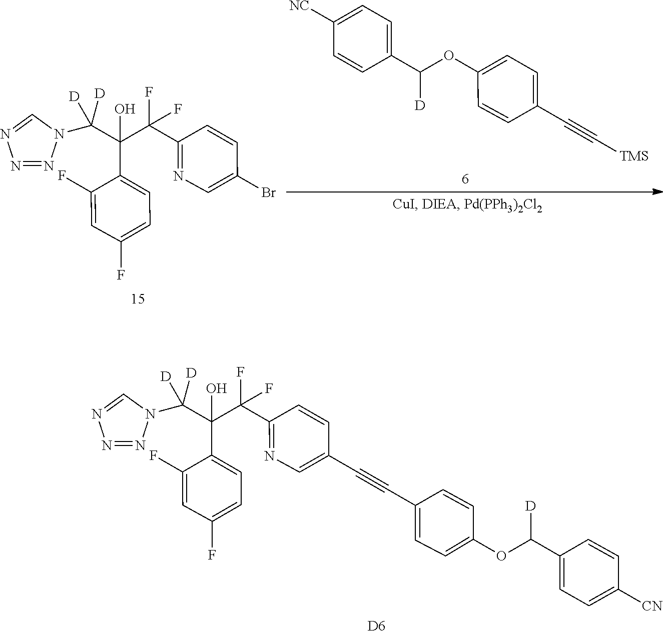 [SMILES: [2H]C([2H])(n1cnnn1)C(O)(c1ccc(F)cc1F)C(F)(F)c1ccc(Br)cn1 ; [2H]C(Oc1ccc(C#C[Si](C)(C)C)cc1)c1ccc(C#N)cc1 ; [2H]C(Oc1ccc(C#Cc2ccc(C(F)(F)C(O)(c3ccc(F)cc3F)C([2H])([2H])n3cnnn3)nc2)cc1)c1ccc(C#N)cc1 ; "D6"]

Compound 15 (1 mol), as well as compound 6 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D6.

¹H NMR (300 MHz, DMSO) δ 9.14 (s, 1H), 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.28 (s, 1H).

### Example 7: Synthesis of Compound D7

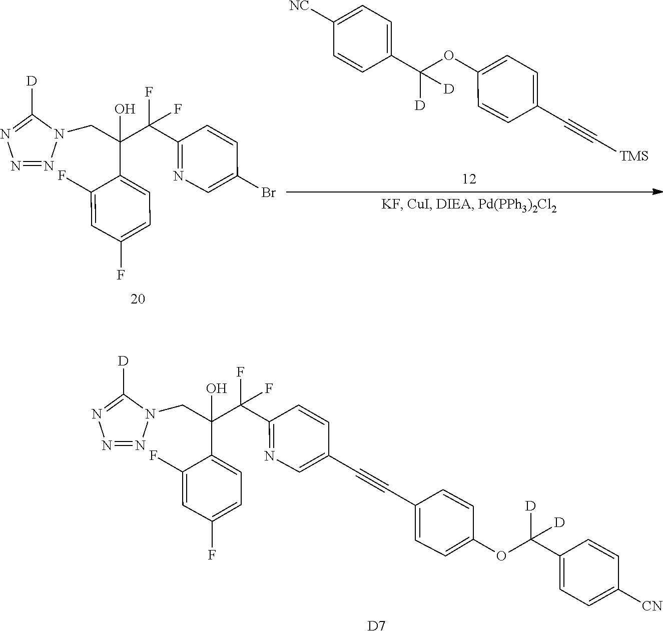 [SMILES: [2H]c1nnnn1CC(O)(c1ccc(F)cc1F)C(F)(F)c1ccc(Br)cn1 ; [2H]C([2H])(Oc1ccc(C#C[Si](C)(C)C)cc1)c1ccc(C#N)cc1 ; [2H]c1nnnn1CC(O)(c1ccc(F)cc1F)C(F)(F)c1ccc(C#Cc2ccc(OC([2H])([2H])c3ccc(C#N)cc3)cc2)cn1 ; "D7"]

Compound 20 (1 mol), as well as compound 12 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D7.

¹H NMR (300 MHz, DMSO) δ 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.65-5.60 (d, J=15.0 Hz, 1H), 5.12-5.07 (d, J=15.0 Hz, 1H).

### Example 8: Synthesis of Compound D8

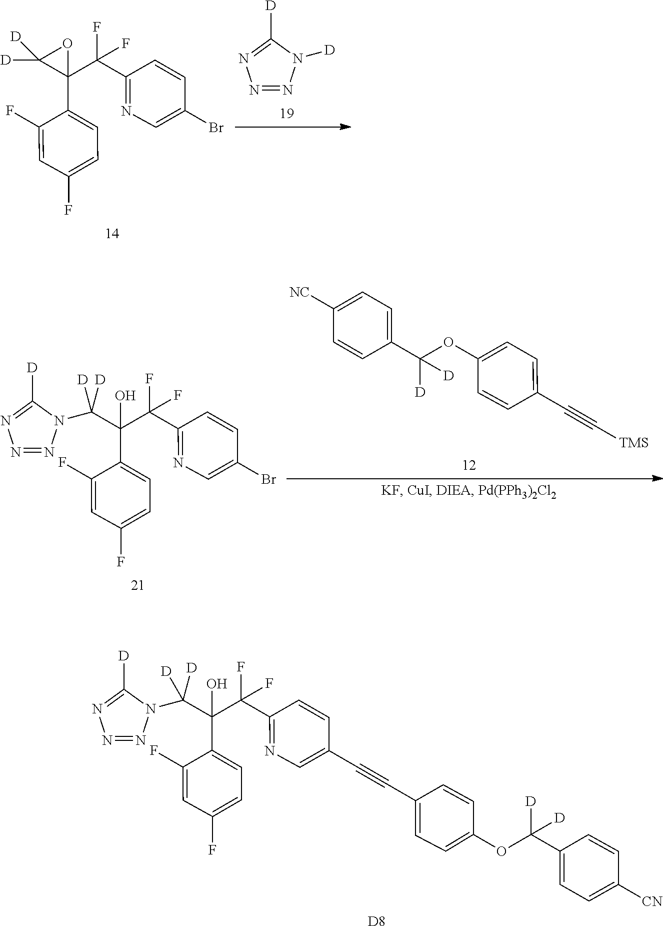

Step 1: compounds 14 (2 mmol) and 19 (6 mmol) were dissolved in N, N-dimethylformamide, and potassium carbonate (6 mmol) was then added to react at 90° C. After the reaction was complete, the reaction solution was poured in ice water and extracted 3 times with ethyl acetate; after being dried, the extract was spin-dried and subjected to column chromatography to obtain compound 21.

Step 2: Compound 21 (1 mol), as well as compound 12 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D8.

¹H NMR (300 MHz, DMSO) δ 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.58-7.55 (d, J=9.0 Hz, 2H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H).

### Example 9: Synthesis of Compound D9

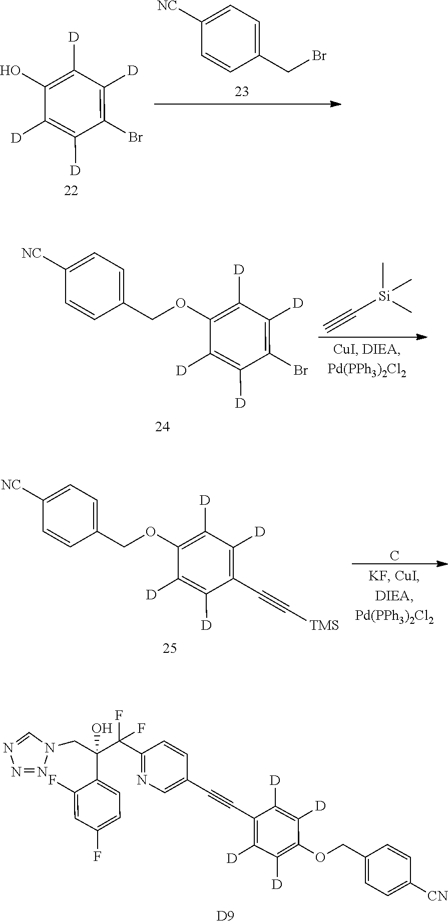

Step 1: compounds 22 (2 mmol) and 23 (2 mmol) were dissolved in N, N-dimethylformamide, and potassium carbonate (4 mmol) was then added to react at 50° C. After the reaction was complete, the reaction solution was poured in ice water and extracted 3 times with ethyl acetate; after being dried, the extract was spin-dried and subjected to column chromatography to obtain compound 24.

Step 2: Compound 24 (2 mmol), ethynyltrimethylsilane (4 mmol), CuI (5% mol), Pd(PPh₃)₂Cl₂ (10% mol), and N, N-diisopropylethylamine (10 mmol) were dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain compound 25.

Step 3: Compound C (1 mol), as well as compound 25 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D9.

¹H NMR (300 MHz, DMSO) δ 9.14 (s, 1H), 8.71-8.70 (d, J=3.0 Hz, 1H), 8.07-8.03 (dd, J=9.0, 3.0 Hz, 1H), 7.89-7.87 (d, J=6.0 Hz, 2H), 7.66-7.64 (d, J=6.0 Hz, 2H), 7.49-7.46 (d, J=9.0 Hz, 1H), 7.32 (s, 1H), 7.23-7.14 (m, 2H), 6.92-6.86 (td, J=9.0, 3.0 Hz, 1H), 5.65-5.60 (d, J=15.0 Hz, 1H), 5.28 (s, 2H), 5.12-5.07 (d, J=15.0 Hz, 1H).

### Example 10: Synthesis of Compound D10

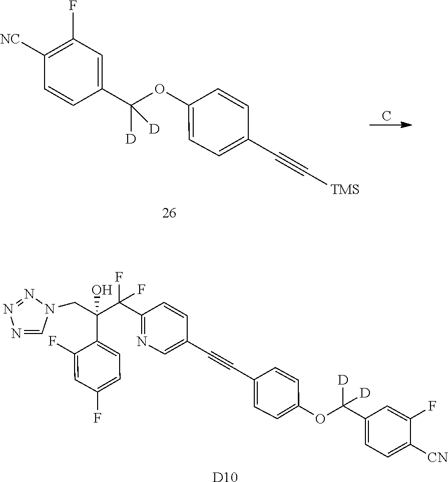

Compound C (1 mol), as well as compound 26 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D10.

¹H NMR (500 MHz, CDCl₃) δ 8.75 (s, 1H), 8.62 (s, 1H), 7.86 (d, J=8.0 Hz, 1H), 7.67 (t, J=8.0 Hz, 1H), 7.53 (d, J=8.0 Hz, 1H), 7.50 (d, J=9.0 Hz, 2H), 7.34-7.32 (m, 4H), 6.95 (d, J=9.0 Hz, 2H), 6.77-6.75 (m, 1H), 6.67-6.65 (m, 1H), 5.59 (d, J=14.0 Hz, 1H), 5.12 (d, J=14.0 Hz, 1H).

### Example 11: Synthesis of Compound D21

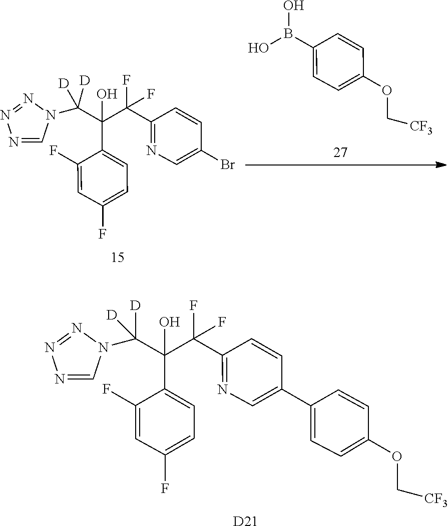 [SMILES: [2H]C([2H])(n1cnnn1)C(O)(c1ccc(F)cc1F)C(F)(F)c1ccc(Br)cn1 ; OB(O)c1ccc(OCC(F)(F)F)cc1 ; [2H]C([2H])(n1cnnn1)C(O)(c1ccc(F)cc1F)C(F)(F)c1ccc(-c2ccc(OCC(F)(F)F)cc2)cn1 ; "D21"]

Compound 15 (1 mol), as well as compound 27 (1 mol), Pd(PPh₃)₂Cl₂ (10% mol), and K₃PO₄, was dissolved in N, N-dimethylformamide and reacted at 80° C. until the raw materials were reacted completely. The reaction solution was poured in ice water and extracted 3 times with ethyl acetate; after being dried, the extract was spin-dried and subjected to column chromatography to obtain the final product D21.

¹H NMR (500 MHz, CDCl₃) δ 8.76 (s, 1H), 8.70 (s, 1H), 7.95 (d, J=8.0 Hz, 1H), 7.70 (s, 1H), 7.64 (d, J=8.5 Hz, 1H), 7.54 (d, J=8.5 Hz, 2H), 7.42-7.37 (m, 1H), 7.08 (d, J=8.5 Hz, 2H), 6.79-6.75 (m, 1H), 6.69-6.66 (m, 1H), 4.44-4.39 (m, 2H).

### Example 12: Synthesis of Compound D22

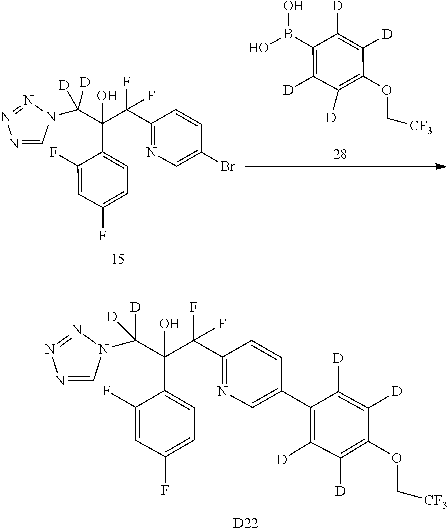 [SMILES: [2H]C([2H])(n1cnnn1)C(O)(c1ccc(F)cc1F)C(F)(F)c1ccc(Br)cn1 ; [2H]c1c([2H])c(B(O)O)c([2H])c([2H])c1OCC(F)(F)F ; [2H]c1c([2H])c(-c2ccc(C(F)(F)C(O)(c3ccc(F)cc3F)C([2H])([2H])n3cnnn3)nc2)c([2H])c([2H])c1OCC(F)(F)F ; "D22"]

Compound 15 (1 mol), as well as compound 28 (1 mol), Pd(PPh₃)₂Cl₂ (10% mol), and K₃PO₄, was dissolved in N, N-dimethylformamide and reacted at 80° C. until the raw materials were reacted completely. The reaction solution was poured in ice water and extracted 3 times with ethyl acetate; after being dried, the extract was spin-dried and subjected to column chromatography to obtain the final product D22.

¹H NMR (500 MHz, CDCl₃) δ 8.76 (s, 1H), 8.70 (s, 1H), 7.95 (d, J=8.0 Hz, 1H), 7.70 (s, 1H), 7.64 (d, J=8.5 Hz, 1H), 7.42-7.37 (m, 1H), 6.79-6.75 (m, 1H), 6.69-6.66 (m, 1H), 4.44-4.39 (m, 2H).

### Example 13: Synthesis of Compound D23

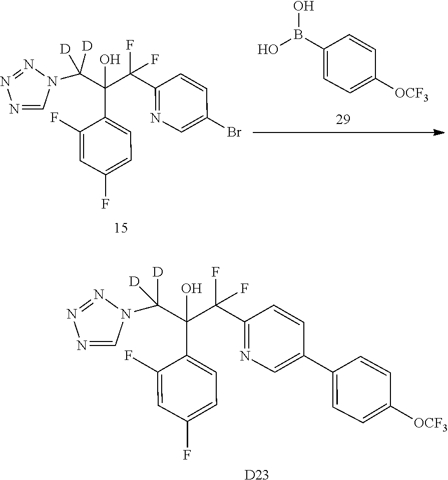 [SMILES: [2H]C([2H])(n1cnnn1)C(O)(c1ccc(F)cc1F)C(F)(F)c1ccc(Br)cn1 ; OB(O)c1ccc(OC(F)(F)F)cc1 ; [2H]C([2H])(n1cnnn1)C(O)(c1ccc(F)cc1F)C(F)(F)c1ccc(-c2ccc(OC(F)(F)F)cc2)cn1 ; "D23"]

Compound 15 (1 mol), as well as compound 29 (1 mol), Pd(PPh₃)₂Cl₂ (10% mol), and K₃PO₄, was dissolved in N, N-dimethylformamide and reacted at 80° C. until the raw materials were reacted completely. The reaction solution was poured in ice water and extracted 3 times with ethyl acetate; after being dried, the extract was spin-dried and subjected to column chromatography to obtain the final product H23.

¹H NMR (500 MHz, CDCl₃) δ 8.76 (s, 1H), 8.70 (s, 1H), 7.97 (dd, J=8.0, 2.0 Hz, 1H), 7.68 (d, J=8.5 Hz, 1H), 7.60-7.56 (m, 3H), 7.43-7.36 (m, 3H), 6.80-6.76 (m, 1H), 6.70-6.67 (m, 1H).

### Example 14: Synthesis of Compound D24

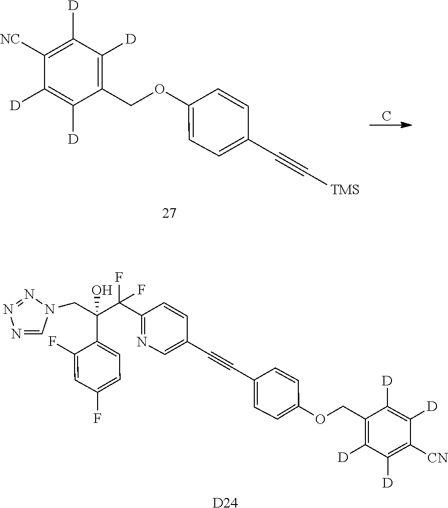

Compound C (1 mol), as well as compound 27 (1 mol), CuI (5% mol), Pd(PPh₃)₂Cl₂ (10% mol), N, N-diisopropylethylamine (5 mol), and KF (1 mmol), was dissolved in N, N-dimethylformamide and reacted at 60° C. until the raw materials were reacted completely. The reaction solution was poured in ice water and extracted 3 times with ethyl acetate; after being dried, the extract was spin-dried and subjected to column chromatography to obtain the final product D24.

¹H NMR (500 MHz, DMSO) δ 9.14 (s, 1H), 8.71-8.70 (d, J=3.0 Hz, 1H), 8.07-8.03 (dd, J=9.0, 3.0 Hz, 1H), 7.49-7.46 (d, J=9.0 Hz, 1H), 7.32 (s, 1H), 7.23-7.14 (m, 2H), 7.12-7.09 (d, J=9.0 Hz, 2H), 6.92-6.86 (td, J=9.0, 3.0 Hz, 1H), 5.65-5.60 (d, J=15.0 Hz, 1H), 5.28 (s, 2H), 5.12-5.07 (d, J=15.0 Hz, 1H).

### Example 15: Synthesis of Compound D25

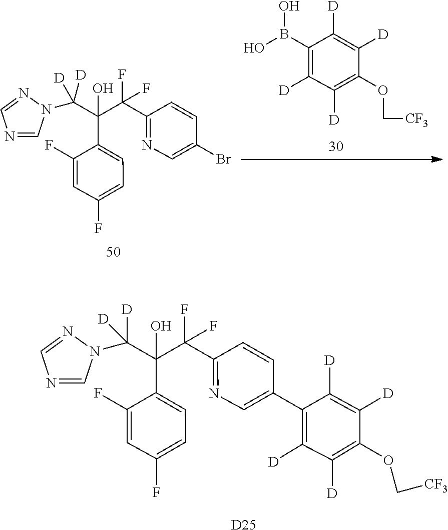 [SMILES: [2H]C([2H])(n1cncn1)C(O)(c1ccc(F)cc1F)C(F)(F)c1ccc(Br)cn1 ; [2H]c1c([2H])c(B(O)O)c([2H])c([2H])c1OCC(F)(F)F ; [2H]c1c([2H])c(-c2ccc(C(F)(F)C(O)(c3ccc(F)cc3F)C([2H])([2H])n3cncn3)nc2)c([2H])c([2H])c1OCC(F)(F)F ; "D25"]

Compound 15 (1 mol), as well as compound 30 (1 mol), Pd(PPh₃)₂Cl₂ (10% mol), and K₃PO₄ (2 mmol), was dissolved in N, N-dimethylformamide and reacted at 80° C. until the raw materials were reacted completely. The reaction solution was poured in ice water and extracted 3 times with ethyl acetate; after being dried, the extract was spin-dried and subjected to column chromatography to obtain the final product D25.

¹H NMR (500 MHz, CDCl3) δ 8.72 (s, 1H), 8.16 (s, 1H), 7.92 (dd, J=8.5, 2.0 Hz, 1H), 7.69 (s, 1H), 7.61 (d, J=8.0 Hz, 1H), 7.52-7.47 (m, 1H), 6.77-6.70 (m, 1H), 4.42 (q, J=8.0 Hz, 2H).

### Example 16: Synthesis of Compound G

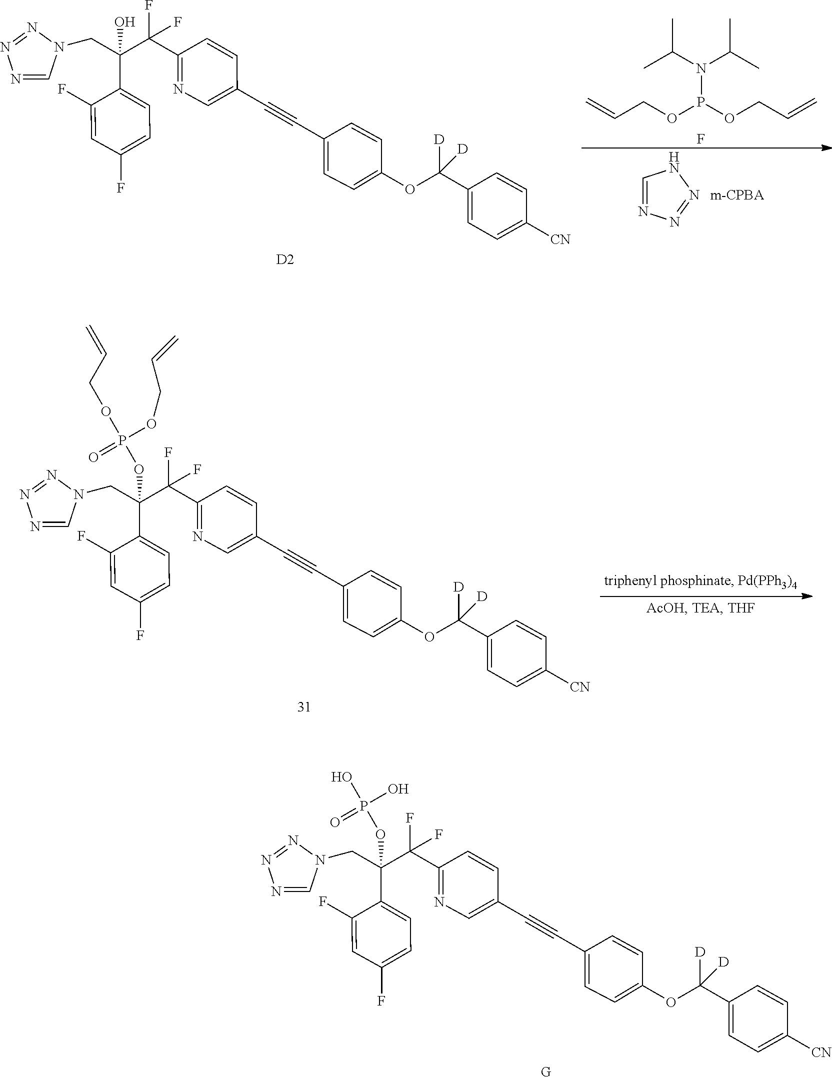

Step 1: compound D2 (1 mmol) and 1-H-tetrazolium (5 mmol) were dissolved in dichloromethane (10 mL), and the dichloromethane solution of compound F (4 mmol) was then added to the system dropwise; stirring at room temperature, reaction was performed for 2 hours and then cooled to −5° C.; the dichloromethane solution of m-CPBA (4 mmol) was added dropwise to the system and then the reaction was performed for 1 hour at −5° C.; the reaction system was added with 50 mL of dichloromethane, then washed twice with 5% sodium thiosulfate, twice with 10% sodium bicarbonate, and twice with the saturated aqueous solution of sodium chloride; after drying with anhydrous sodium sulfate, spin-drying was performed. After reversed-phase preparation, the product was lyophilized in vacuo to obtain compound 31.

Step 2: compound 31 (0.5 mmol) was dissolved in tetrahydrofuran, and triphenyl phosphinate (0.5 mmol), tetratriphenylphosphine palladium (0.05 mmol), triethylamine (1 mmol), and 1M acetic acid (2.5 mmol) were added in sequence under an ice-water condition to react overnight at room temperature; the solid was filtered off, and the filtrate was spin-dried. After reversed-phase preparation, the product was lyophilized in vacuo to obtain compound G.

¹H NMR (400 MHz, CD₃OD) δ 9.33 (s, 1H), 8.70 (s, 1H), 7.92 (d, J=8.0 Hz, 1H), 7.75 (d, J=8.4 Hz, 2H), 7.64 (d, J=8.4 Hz, 2H), 7.51 (d, J=8.0 Hz, 1H), 7.45-7.39 (m, 1H), 7.34-7.27 (m, 1H), 7.05 (d, J=8.0 Hz, 2H), 6.95-6.89 (m, 1H), 6.83-6.78 (m, 1H), 6.21 (d, J=15.2 Hz, 1H), 5.94 (d, J=15.2 Hz, 1H). 31P NMR (400 MHz, CD₃OD) δ −6.98 (s).

### Example 17: Synthesis of Chiral Compound 33

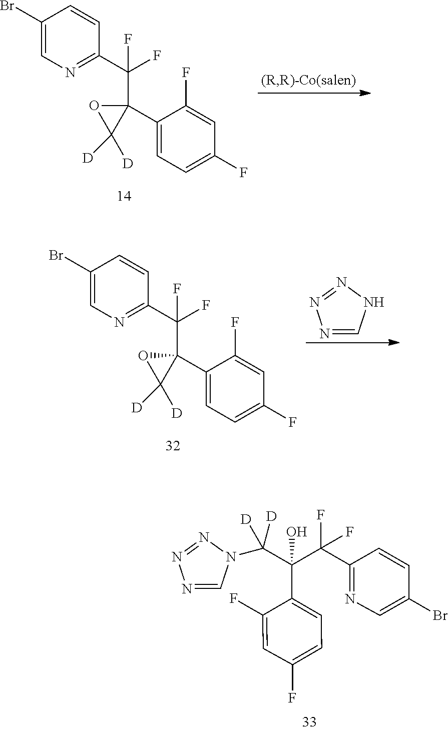 [SMILES: [2H]C1([2H])OC1(c1ccc(F)cc1F)C(F)(F)c1ccc(Br)cn1 ; [2H]C1([2H])O[C@@]1(c1ccc(F)cc1F)C(F)(F)c1ccc(Br)cn1 ; c1nnn[nH]1 ; [2H]C([2H])(n1cnnn1)[C@](O)(c1ccc(F)cc1F)C(F)(F)c1ccc(Br)cn1]

Step 1: (R, R) —Co (salen) (0.3 mmol) was dissolved in toluene, acetic acid (13 mmol) was then added to react at room temperature for 30 minutes, and the reaction solution was spin-dried. Compound 14 (15 mmol) and the formed catalyst were dissolved in toluene, water (8 mmol) was then added dropwise under an ice-bath condition, and reaction was performed at room temperature for 14 hours. The reaction solution was spin-dried and subjected to column chromatography to obtain compound 32.

Step 2: compound 32 (2 mmol) and 1-H-tetrazole (6 mmol) were dissolved in N, N-dimethylformamide, and potassium carbonate (6 mmol) was then added to react at 90° C. After the reaction was complete, the reaction solution was poured in ice water and extracted 3 times with ethyl acetate; after being dried, the extract was spin-dried and subjected to column chromatography to obtain compound 33.

¹H NMR (500 MHz, CDCl₃) δ 8.74 (s, 1H), 8.62 (s, 1H), 7.94 (d, J=7.5 Hz, 1H), 7.46 (d, J=9.0 Hz, 1H), 7.31-7.26 (m, 1H), 6.88 (s, 1H), 6.78-6.74 (m, 1H), 6.70-6.67 (m, 1H).

### Example 18: Synthesis of p-Toluenesulfonate of Compound D2

D2 (5 mmol) was dissolved in isopropyl acetate, p-toluenesulfonic acid monohydrate (5 mmol) was then added at 30° C. to react at 50-60° C. for 2 hours until a solid precipitates. After stirring for 10 hours, the solid was filtered with suction, and the filter cake was washed with a small amount of isopropyl acetate and then dried in a vacuum drying box to obtain a p-toluenesulfonate of compound D2.

The specific deuterated azole compounds included in the invention are as follows:

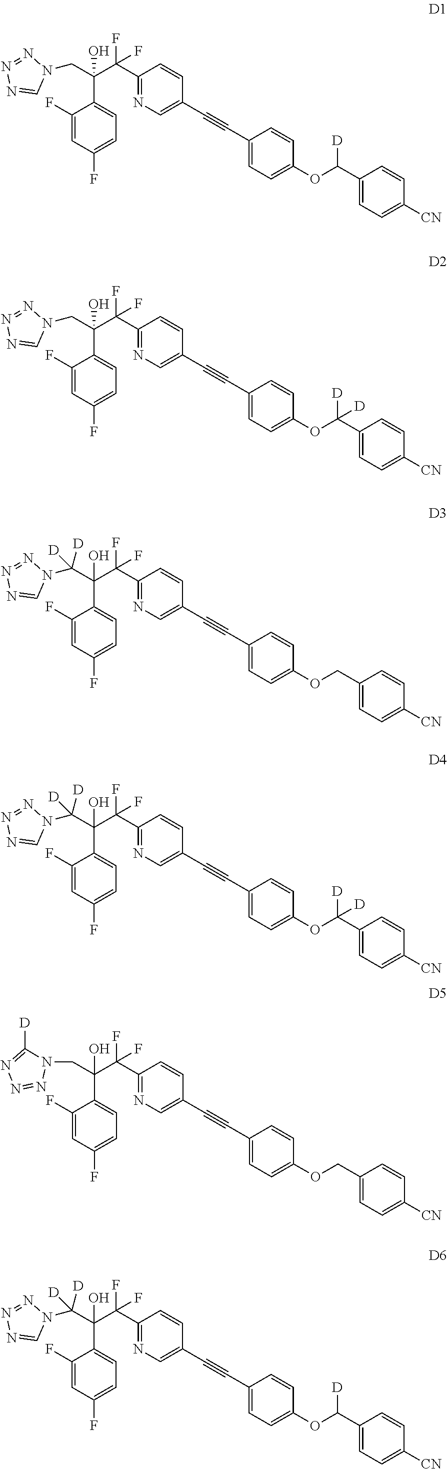

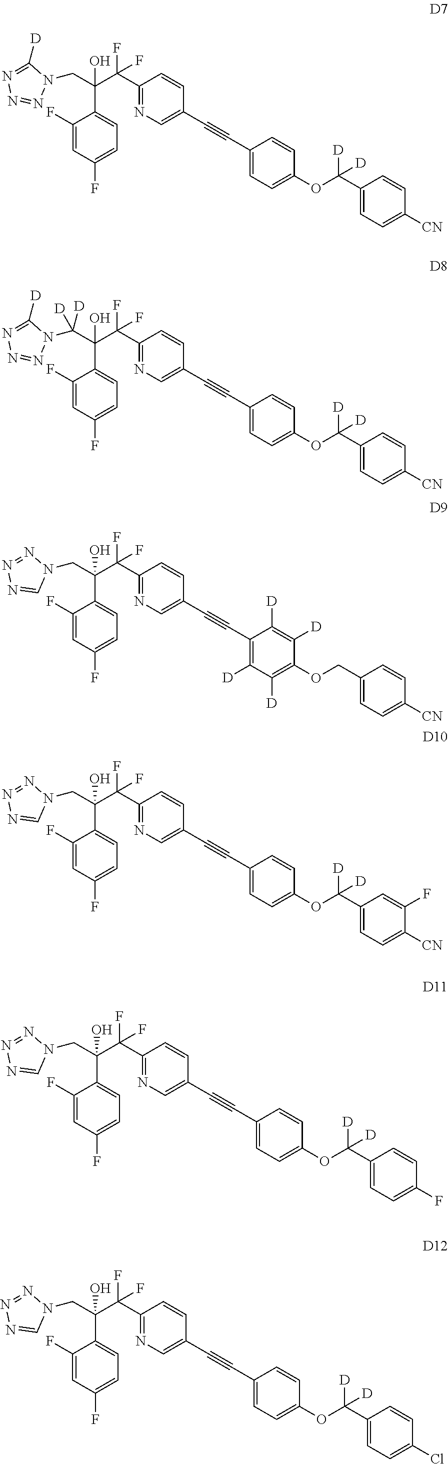

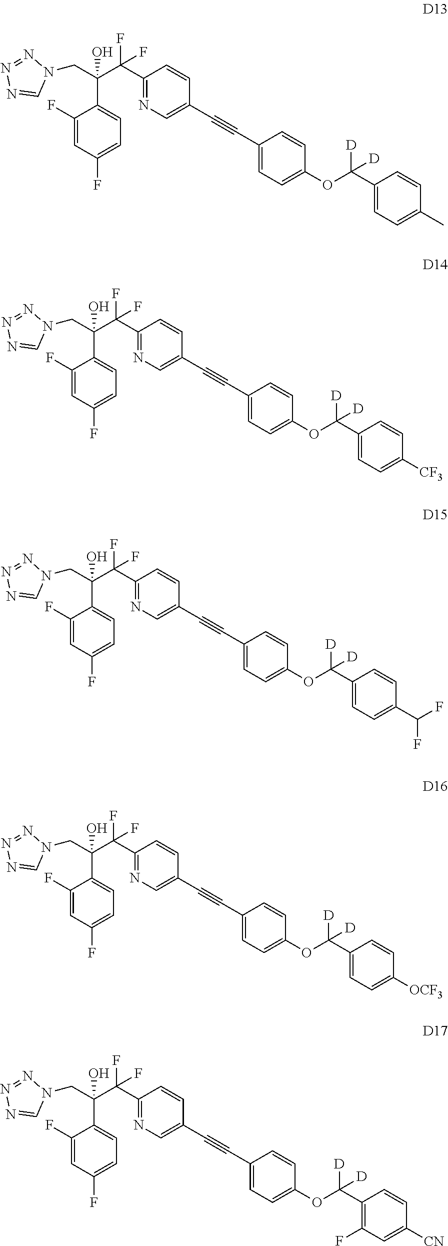 [SMILES: [2H]C([2H])(Oc1ccc(C#Cc2ccc(C(F)(F)[C@](O)(Cn3cnnn3)c3ccc(F)cc3F)nc2)cc1)c1ccc(C)cc1 ; [2H]C([2H])(Oc1ccc(C#Cc2ccc(C(F)(F)[C@](O)(Cn3cnnn3)c3ccc(F)cc3F)nc2)cc1)c1ccc(C(F)(F)F)cc1 ; [2H]C([2H])(Oc1ccc(C#Cc2ccc(C(F)(F)[C@](O)(Cn3cnnn3)c3ccc(F)cc3F)nc2)cc1)c1ccc(C(F)F)cc1 ; [2H]C([2H])(Oc1ccc(C#Cc2ccc(C(F)(F)[C@](O)(Cn3cnnn3)c3ccc(F)cc3F)nc2)cc1)c1ccc(OC(F)(F)F)cc1 ; [2H]C([2H])(Oc1ccc(C#Cc2ccc(C(F)(F)[C@](O)(Cn3cnnn3)c3ccc(F)cc3F)nc2)cc1)c1ccc(C#N)cc1F]

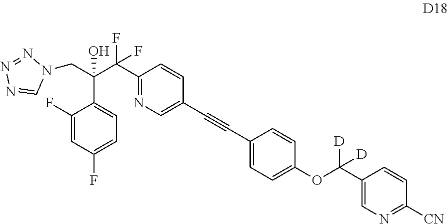 [SMILES: [2H]C([2H])(Oc1ccc(C#Cc2ccc(C(F)(F)[C@](O)(Cn3cnnn3)c3ccc(F)cc3F)nc2)cc1)c1ccc(C#N)nc1]

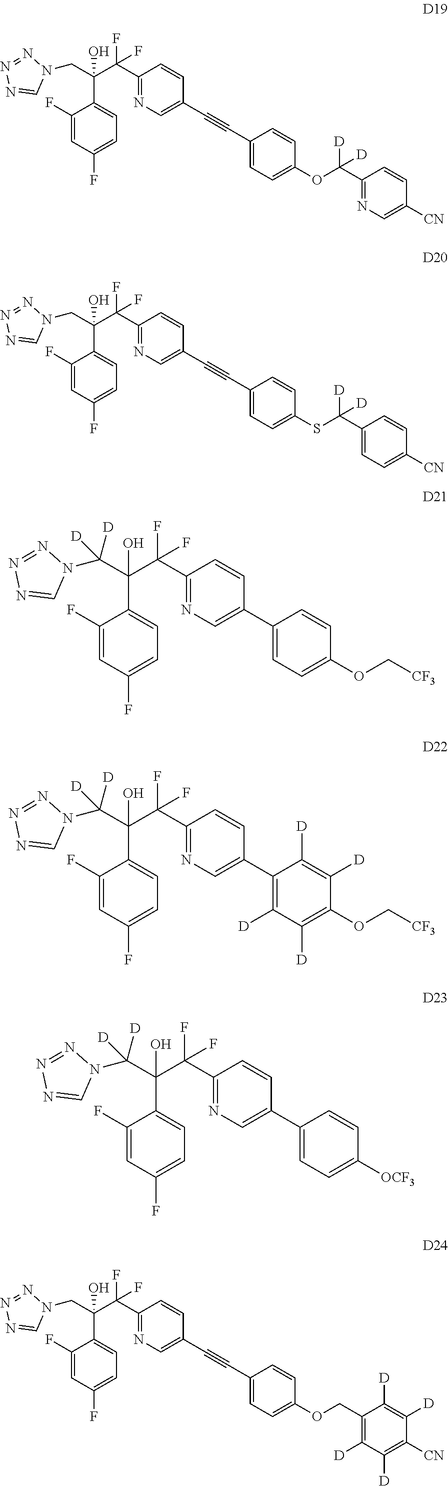

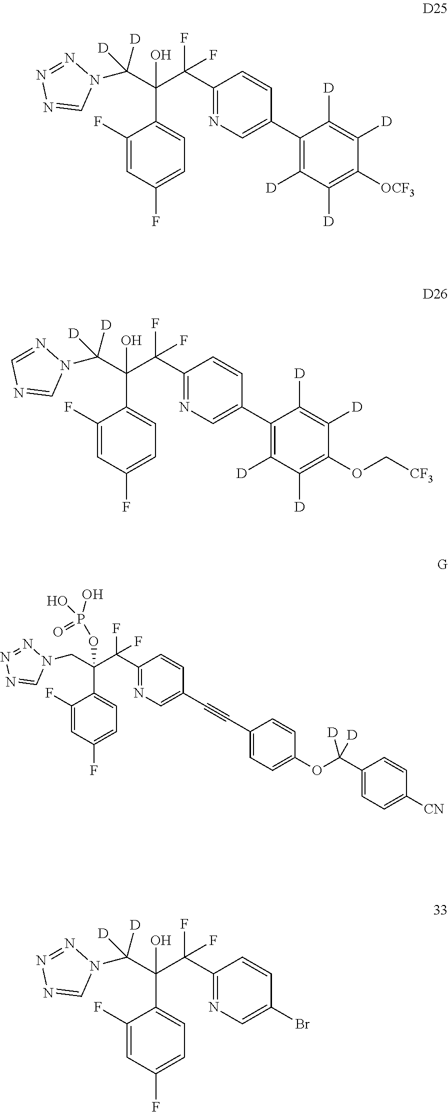 [SMILES: [2H]c1c([2H])c(-c2ccc(C(F)(F)C(O)(c3ccc(F)cc3F)C([2H])([2H])n3cnnn3)nc2)c([2H])c([2H])c1OC(F)(F)F ; [2H]c1c([2H])c(-c2ccc(C(F)(F)C(O)(c3ccc(F)cc3F)C([2H])([2H])n3cncn3)nc2)c([2H])c([2H])c1OCC(F)(F)F ; [2H]C([2H])(Oc1ccc(C#Cc2ccc(C(F)(F)[C@@](Cn3cnnn3)(OP(=O)(O)O)c3ccc(F)cc3F)nc2)cc1)c1ccc(C#N)cc1 ; [2H]C([2H])(n1cnnn1)C(O)(c1ccc(F)cc1F)C(F)(F)c1ccc(Br)cn1]

### Experimental Example 1

In vitro bacteriostatic experiment of the compounds of the invention

(I) Experimental method: The conventional in vitro bacteriostatic experiment method was adopted (for details, see: Antimicrob Agents Chemother 1995, 39 (5): 1169).

(I) Materials and Methods

(1) Experimental Strain

The fungal strain selected for this experiment was provided by the Fungal Department of Shanghai Changzheng Hospital (or purchased from the Institute of Materia Medica, Chinese Academy of Sciences).

Candida albicans (standard strain SC5314)

(2) Test Method

Preparation of bacterial suspension: The above fungi were cultured in YEPD liquid medium at 35° C. for 16 hours, activated twice, counted with a blood cell counting plate, and the bacteria concentration was adjusted to 1*10⁴ to 1*10⁵ cells/mL with RPM1640 liquid medium.

Preparation of drug solution: The test compound of the invention was dissolved in dimethyl sulfoxide to prepare a 0.8 mg/mL drug storage solution, and the 0.8 mg/mL drug storage solution was diluted to 8 μg/mL with RPM1640 before the experiment.

Inoculation: RPM1640 100 μL was added as a blank control in well 1 of the 96-well plate; 100 μL of the bacterial suspension was added in each of wells 3-12, 200 μL of the bacterial suspension and 2 μL of the drug solution were added in well 2 and the drug solution in wells 2-11 was diluted 10 folds, and the drug concentrations of these wells were 8, 4, 2, 1, 0.5, 0.25, 0.125, 0.0625, 0.0313, and 0.0156 μg/mL, respectively. No drug solution was added in well 12 to make positive control. The drug control was fluconazole.

(II) Test Results

The results of the in vitro bacteriostatic experiment are shown in Table 1.

TABLE 1
Minimum in vitro antifungal concentration
of target compounds (MIC₈₀, μg/mL)

| Compound No. | Candida albicans SC5314 |
|---|---|
| D1 | 0.0156 |
| D2 | 0.0156 |
| D3 | 0.0313 |
| D5 | 0.0156 |
| VT-1598 | 0.0156 |
| Fluconazole | 0.5 |

The above experimental results show that the compounds of the invention have better antifungal activity, and the in vitro antibacterial activity of the compounds was significantly stronger than that of fluconazole; and compared with VT-1598, the in vitro antibacterial activity was hardly affected after deuteration.

### Experimental Example 2

In vitro human liver microsomal enzyme stability test

(1) Compound Information

| Sample | | | Content/ | Conversion | Storage |
|---|---|---|---|---|---|
| for test | MW | FW | purity | factor | condition |
| VT-1598 | 584.52 | NA | >95% | 1.0 | Room |
| | | | | | temperature |
| D2 | 586.54 | NA | >95% | 1.0 | Room |
| | | | | | temperature |

(2) Microsomes

The human mixed liver microsomes used in this experiment were sourced from Corning Company in the United States or other commonly available commercial companies and stored at −90° C. to −60° C.

(3) Experimental Procedure

The test compound will be co-incubated with human liver microsomes under the following conditions (see table). The test compound was added to the incubation tube to obtain a solution, and the mixture was immediately separated and placed in a 37° C. water bath. Then the working solution of NADPH was added to start the reaction. A portion of the incubation solution was taken out at 0, 5, 10, 20, 40, and 60 minutes and transferred to acetonitrile containing an internal standard to terminate the reaction. After protein precipitation, the solution was centrifuged at 3700 rpm for 10 minutes and the supernatant was collected. Test compounds in the supernatant were analyzed by LC-MS/MS method. The in vitro intrinsic elimination rates were calculated based on the elimination half-life of the test compounds in the incubation system. Midazolam was incubated in parallel as a positive control. The incubation conditions were summarized in the following table (the content of organic solvents in the incubation system does not exceed 1%), and the test compounds and positive control were both incubated in parallel in two copies:

| | |
|---|---|
| Microsome | 0.5 mg · mL⁻¹ (test compound) |
| | 0.2 mg · mL⁻¹ (midazolam) |
| Incubation buffer | Phosphate buffer (100 mM, pH 7.4) |
| Starting concentration | 1 μM |
| of test compound for | |
| incubation | |
| Final volume of | 0.2 mL (test compound) |
| incubation system | 0.1 mL (midazolam) |
| Incubation time | 0, 5, 10, 20, 40, and 60 min (test compound) |
| | 0, 5, and 20 min (midazolam) |
| Magnesium chloride | 3 mM |
| NADPH | 1 mM |
| Parallel reaction | Two parallel copies |

(4) Data Analysis

The analyte/internal standard peak area ratio (Aanalyte/AIS) will be calculated by the instrument, and the remaining percentage (% Control) was calculated from the Aanalyte/AIS ratios in the non-zero time sample and the zero time sample. The change of Ln (% Control) versus incubation time was plotted and linear fitting was performed. The clearance constant (k, min-¹), clearance half-life (T_{1/2}, min), and in vitro intrinsic clearance (CLint, μL×min⁻¹×mg⁻¹ proteins) of test compounds were calculated from the following equations.

k=−slope

T1/2=0.693/k

CLint=k/Cprotein

Cprotein (m×gmL⁻¹) refers to the microsomal protein concentration in the incubation system.

(5) The Results are Shown in Table 2

TABLE 2
Stability tests of target compounds
on human liver microsomal enzymes

| | | | | CL_{int, microsome} |
|---|---|---|---|---|
| | | k | T_{1/2} | (μL × min⁻¹ × mg⁻¹ |
| Compound | Species | (min⁻¹) | (min) | proteins) |
| VT-1598 | Human | 0.00530 | 140 | 10.6 |
| D2 | | 0.00176 | 517 | 3.52 |
| Midazolam | | 0.0548 | 12.7 | 274 |

It can be seen from the results that the deuterated compound D2 has significantly better stability to human liver microsomal enzymes than VT-1598, and deuterated VT-1598 has a broad market prospect.

In summary, the compound of the invention has a good inhibitory activity on the human pathogenic fungus Candida albicans, and the stability of the compound is significantly improved. The pharmacodynamic and pharmacokinetic properties of compound are significantly improved.

While the invention has been described in detail with reference to the aforesaid preferred embodiments, it should be appreciated that the foregoing description should not be construed as limiting the invention. Various modifications and substitutions will be apparent to those skilled in the art upon reading the foregoing contents. Accordingly, the scope of the invention should be defined by the appended claims.

1. A deuterated azole compound or a pharmaceutically acceptable salt thereof, being represented by formula (I): 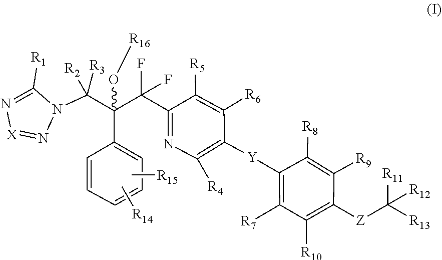
wherein,
R1-R12 are each selected from a hydrogen atom or a deuterium atom;
R13 is selected from C1-C6 alkyl, halogen-substituted alkyl, phenyl, pyridyl, C1-6 alkyl-substituted phenyl or pyridyl, halogen-substituted C1-6 alkyl-substituted phenyl or pyridyl, halogen-substituted phenyl or pyridyl, nitro-substituted phenyl or pyridyl, cyano-substituted phenyl or pyridyl, and trifluoromethyl-substituted phenyl or pyridyl, respectively;
R14 and R15 are each selected from a deuterium atom, a hydrogen atom, or a halogen;
R16 is a hydrogen atom or a phosphate group;
X represents N or CH;
Y represents 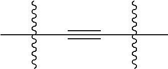 [SMILES: *C#C*]
or non-substituted; and
Z represents O or S.

2. The compound or the pharmaceutically acceptable salt thereof according to claim 1, wherein the compound is a compound represented by formula (II) or a compound represented by formula (III): 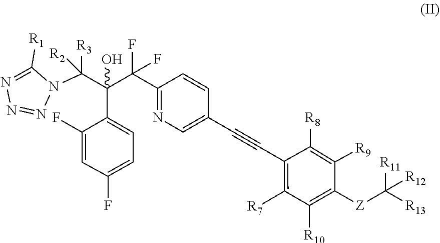
wherein R1-R12 are each selected from a hydrogen atom or a deuterium atom;
R13 is selected from toluene, cyano-substituted phenyl, trifluoromethyl-substituted phenyl, difluoromethyl-substituted phenyl, trifluoromethoxy-substituted phenyl, halogen-substituted phenyl, and cyano-substituted pyridine, and Z is an oxygen atom or a sulfur atom; 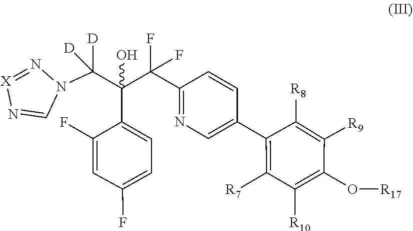
wherein R7-R10 are each selected from a hydrogen atom or a deuterium atom, and R17 is a trifluoromethyl group or a trifluoromethoxy group.

3. The compound or the pharmaceutically acceptable salt thereof according to claim 2, wherein the phenyl or pyridyl in the R13 group is a deuterated phenyl or a deuterated pyridyl.

4. The compound or the pharmaceutically acceptable salt thereof according to claim 1, wherein the compound includes R-isomers or S-isomers;
the R-isomers are represented by: 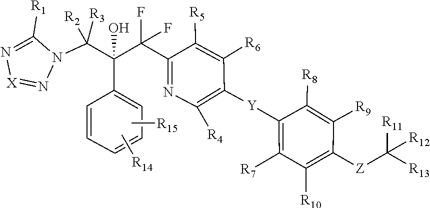
the S-isomers are represented by:

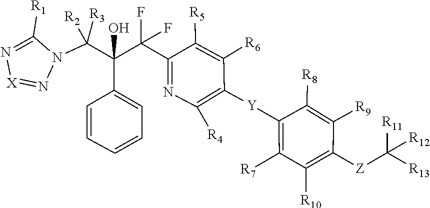

5. The compound or the pharmaceutically acceptable salt thereof according to claim 4, wherein the pharmaceutically acceptable salt is an inorganic acid salt or an organic acid salt; and
the inorganic acid salt is hydrochloride, and the organic acid salt is p-toluenesulfonate.

6. A crystalline compound, being the compound or the pharmaceutically acceptable salt thereof according to claim 1.

7. A preparation method of the deuterated azole compound according to claim 1, being shown as Route (1): 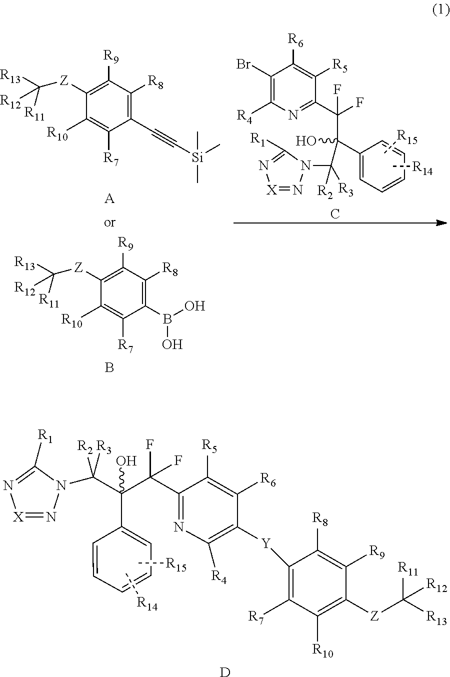
the method specifically comprising:
dissolving compound C in an organic solvent, adding a palladium catalyst, an alkali reagent, and compound A or compound B, and heating in the presence of nitrogen to cause a reaction to obtain a target product, i.e., compound D.

8. A pharmaceutical composition, comprising the deuterated azole compound or the pharmaceutically acceptable salt thereof according to claim 1.

9. The pharmaceutical composition according to claim 8, comprising: at least one pharmaceutically acceptable carrier, or/and at least one additional antifungal compound.

10. An application of the pharmaceutical composition according to claim 8 in preparing drugs for treating fungal infections.
